(19) United States
(12) Patent Application Publication
(10) Pub. No.: US 2010/0076304 A1
(43) Pub. Date: Mar. 25, 2010

(54) INVISIBLE LIGHT IRRADIATION APPARATUS AND METHOD FOR CONTROLLING INVISIBLE LIGHT IRRADIATION APPARATUS

(75) Inventor: Yuichi TERAMURA, Ashigarakami-gun (JP)

Correspondence Address: MCGINN INTELLECTUAL PROPERTY LAW GROUP, PLLC, 8321 OLD COURTHOUSE ROAD, SUITE 200, VIENNA, VA, 22182-3817, US

(73) Assignee: FUJIFILM CORPORATION, Tokyo (JP)

(21) Appl. No.: 12/558,338

(22) Filed: Sep. 11, 2009

(30) Foreign Application Priority Data
Sep. 25, 2008 (JP) JP2008-245324

(51) Int. Cl. A61B 6/00; G01J 5/00

(52) U.S. Cl. 600/425; 250/492.1; 250/330

(57) ABSTRACT

Provision of an invisible light irradiation apparatus in which aiming light can always be recognized in an observation portion image in a normal light observation mode and a special light observation mode, and a method for controlling the invisible light irradiation apparatus. An invisible light irradiation apparatus comprising: an invisible light source which emits invisible light for irradiation of an object to be irradiated; and a guide light source which emits guide light for being combined with the invisible light to irradiate the object to be irradiated to recognize the position of irradiation of the invisible light, wherein a plurality of the guide light sources which emit the guide light in different wavelength regions are provided.

### BACKGROUND OF THE INVENTION

1. Field of the Invention

The present invention relates to an invisible light irradiation apparatus and a method for controlling the invisible light irradiation apparatus, and particularly to a technique for guide light (aiming light) for recognizing the position of irradiation of invisible light.

1. Description of the Related Art

Optical coherence tomography (OCT) is a method for noninvasively measuring a tomographic image of a living body. It is applied to every part of the living body, such as an eye and a cardiovascular system, and used for distinction between a normal portion and a lesion. In the field of digestive organs, a method for diagnosing the invasion depth of a lesion by using optical coherence tomography combined with an endoscope is proposed. In other words, a method is proposed in which the surface of a digestive organ is observed with an endoscope to extract a lesion, and a tomographic image of the part is observed by OCT to determine how deep the lesion reaches and decide the treatment policy. In OCT, to obtain information on a deeper part, infrared light in a range of 800 to 1500 nm which is not much absorbed by water and hemoglobin contained in a living body is used for the light source. This wavelength region is an invisible region, so that where on an object to be measured is irradiated with the measurement light cannot be visually recognized. Therefore, there has been a problem that whether the lesion is exposed to the OCT measurement light or not cannot be recognized, for example, with an endoscope.

Japanese Patent Application Laid-Open No. 11-56772 discloses, in an optical tomographic imaging apparatus in which an optical scanning probe is inserted into the forceps channel of an endoscope, and low coherent infrared light is emitted to an analyte from the optical scanning probe for measurement, a technique in which the position of measurement with low coherent infrared light which cannot be visually recognized can be visually recognized with aiming light in an observation portion image in a monitor obtained by combining the low coherent infrared light with the aiming light (guide light), which is red visible light, and irradiating the test body with white illumination light from the tip portion of the endoscope.

In this manner, the test body is irradiated with illumination light from the tip portion of the endoscope so that an observer can recognize the state of the test body in the observation portion image in the monitor. At this time, only monochromatic light of aiming light which is red visible light is used in Japanese Patent Application Laid-Open No. 11-56772.

Also, in recent endoscopes, as a method for extracting a lesion with higher precision, the so-called special light observation is performed, for example, an object to be measured is observed using for illumination light light having a wavelength in a specific region, and an object to be measured is observed by obtaining only light having a wavelength in a specific region, in the reflected light of illumination light, and displaying an observation portion image in a monitor.

As one example of such a special light observation mode, for example, Japanese Patent No. 4067358 discloses a technique in which a test body is irradiated with light of cyan which is the complementary color of red abundant in a gastric mucosa, separately from conventional illumination light, to highlight blood vessels. Also, Japanese Patent No. 3894761 discloses a technique in which a red cut filter which cuts light having a wavelength of 630 nm or more is used to improve the visibility of blood vessels and the like. Also, Japanese Patent No. 3441449 discloses, in an endoscope apparatus in which the observation of a living body mucosa is performed by an infrared electronic scope in which infrared observation is possible, a technique in which two infrared lights having a wavelength of 790 to 820 nm and a wavelength of 905 to 970 nm are irradiated, and observation portion images of these infrared images are displayed in a monitor.

### SUMMARY OF THE INVENTION

As described above, in the special light observation mode, an observation portion image in the monitor is obtained using light in a wavelength region different from that in a normal light observation mode. Therefore, if the aiming light is monochromatic light, the aiming light may not be visually recognized in the observation portion image in the monitor, depending on the wavelength region of the aiming light, in the special light observation mode, even if the aiming light can be visually recognized in the observation portion image in the monitor in the normal light observation mode.

In Japanese Patent Application Laid-Open No. 11-56772, the aiming light is monochromatic light of only red light. The wavelength of red light is not much absorbed by water, penetrates to a deeper part, and is reflected. Usually, with an endoscope, the wavelength of red light is a suitable for the aiming light, depending on the affected part, but if an object to be measured is observed with an endoscope with a special light observation mode in which red light having the same wavelength as the aiming light is removed, the aiming light may not be visually recognized in the observation portion image in the monitor.

For example, in Japanese Patent No. 3894761, when red light is used as the aiming light during normal observation, observation is performed, with red light cut, in the special light observation mode, so that red aiming light is not observed in the observation portion image in the monitor in the special light observation mode. Therefore, for example, if green light (510 nm) or blue light (440 nm) is used instead of red light, the position of measurement with low coherent light is easily recognized in the observation portion image in the monitor.

On the other hand, for example, in Japanese Patent No. 4067358, cyan light is irradiated as illumination light in the special light observation mode, so that if aiming light of cyan, or blue or green close to cyan is used as it is, the aiming light may not be visually recognized in the observation portion image in the monitor when the aiming light is used with this type of endoscope. Therefore, it is desired that red light is used as the aiming light in the special light observation mode.

Also, in Japanese Patent No. 3441449, visible aiming light is desired in a normal image which is an image in the normal light observation mode, and infrared aiming light which can be received by the infrared electronic scope is desired in an infrared image which is an image in the special light observation mode.

As described above, Japanese Patent Application Laid-Open No. 11-56772 does not disclose switching the wavelength region of the aiming light between the normal light observation mode and the special light observation mode, nor suggest selecting the wavelength region of the aiming light corresponding to the illumination light in the normal light observation mode and the special light observation mode.

Therefore, the aiming light may not be visually recognized in the observation portion image, depending on the wavelength region of the illumination light, in the normal light observation mode or the special light observation mode.

The present invention has been made in view of such circumstances, and it is an object of the present invention to provide an invisible light irradiation apparatus in which aiming light can always be recognized in an observation portion image in a normal light observation mode and a special light observation mode, and a method for controlling the invisible light irradiation apparatus.

To achieve the above object, a first aspect of the present invention provides an invisible light irradiation apparatus comprising: an invisible light source which emits invisible light for irradiating an object to be irradiated; and a guide light source which emits guide light for being combined with the invisible light to irradiate the object to be irradiated to clearly indicate a position of irradiation of the invisible light, wherein a plurality of the guide light sources which emit the guide light in different wavelength regions are provided.

According to the first aspect, the visibility of the aiming light (guide light) is improved, and the aiming light can be recognized in an observation portion image.

To achieve the above object, a second aspect of the present invention provides the invisible light irradiation apparatus according to the first aspect, further comprising: an illumination device which emits illumination light used for observing the object to be irradiated and emits a plurality of the illumination lights in different wavelength regions; an illumination light switching control portion which switches the wavelength region of the illumination light to switch an observation mode for observation of the object to be irradiated; and a guide light control portion which switches the wavelength region of the guide light, wherein the guide light control portion switches the wavelength region of the guide light according to the observation mode switched by the illumination light switching control portion.

According to the second aspect, the aiming light can always be recognized in the observation portion image, corresponding to the switching of the observation mode in which the wavelength region of the illumination light is switched.

To achieve the above object, a third aspect of the present invention provides the invisible light irradiation apparatus according to the second aspect, wherein the guide light control portion switches the wavelength region of the guide light to be different from the wavelength region of the illumination light.

According to the third aspect, the wavelength region of the guide light is different from the wavelength region of the illumination light, so that the aiming light can always be recognized in the observation portion image.

To achieve the above object, a fourth aspect of the present invention provides the invisible light irradiation apparatus according to the second or third aspect, wherein the guide light control portion switches the wavelength region of the guide light so that a color of the guide light is a complementary color for a color of the illumination light.

According to the fourth aspect, the color of the guide light is the complementary color for the color of the illumination light, so that the visibility of the aiming light is improved.

To achieve the above object, a fifth aspect of the present invention provides the invisible light irradiation apparatus according to the second or third aspect, wherein the guide light control portion switches the wavelength region of the guide light so that the guide light is infrared light when the illumination light is infrared light.

According to the fifth aspect, the aiming light can be recognized in an observation portion image which is an infrared image, even if the illumination light is infrared light.

To achieve the above object, a sixth aspect of the present invention provides the invisible light irradiation apparatus according to the first aspect, further comprising: an illumination device which emits illumination light used for observing the object to be irradiated; a received light switching control portion which switches a wavelength region for receiving reflected light from the object to be irradiated to switch an observation mode for the object to be irradiated; and a guide light control portion which switches the wavelength region of the guide light, wherein the guide light control portion switches the wavelength region of the guide light according to the observation mode switched by the received light switching control portion.

According to the sixth aspect, the aiming light can always be recognized in the observation portion image, corresponding to the switching of the observation mode in which the wavelength region of received reflected light is switched.

To achieve the above object, a seventh aspect of the present invention provides the invisible light irradiation apparatus according to any one of the first to sixth aspects, further comprising: an elongated insertion portion which can be inserted into the object to be irradiated; a light guide device which is inserted into the insertion portion, guides the invisible light to irradiate the object to be irradiated from a tip portion of the insertion portion, and guides reflected light reflected by the object to be irradiated; a coherent light extraction device which allows the reflected light guided by the light guide device and the invisible light to interfere with each other to extract a coherent signal corresponding to interfering coherent light; and a signal processing device which performs signal processing corresponding to the coherent signal and generates a tomographic image of the object to be irradiated in a depth direction.

To achieve the above object, an eighth aspect of the present invention provides a method for controlling an invisible light irradiation apparatus, comprising performing control to switch the wavelength region of guide light for being combined with invisible light for irradiation of an object to be irradiated to irradiate the object to be irradiated to clearly indicate a position of irradiation of the invisible light, according to the wavelength region of illumination light for illumination for observation of the object to be irradiated.

To achieve the above object, a ninth aspect of the present invention provides a method for controlling an invisible light irradiation apparatus, comprising performing control to switch the wavelength region of guide light for being combined with invisible light for irradiation of an object to be irradiated to irradiate the object to be irradiated to clearly indicate a position of irradiation of the invisible light, according to the wavelength region of light received for reflected light from the object to be irradiated.

To achieve the above object, a tenth aspect of the present invention provides a method for controlling an invisible light irradiation apparatus, comprising: combining invisible light for irradiation of an object to be irradiated with guide light; irradiating the object to be irradiated with the combined lights to clearly indicate a position of irradiation of the invisible light by the guide light; and emitting illumination light for observing the object to be irradiated, wherein a wavelength region of the guide light is switched according to a wavelength region of the illumination light.

To achieve the above object, an eleventh aspect of the present invention provides A method for controlling an invisible light irradiation apparatus, comprising: combining invisible light for irradiation of an object to be irradiated with guide light; irradiating the object to be irradiated with the combined lights to clearly indicate a position of irradiation of the invisible light by the guide light; and receiving reflected light from the object to be irradiated, wherein a wavelength region of the guide light is switched according to a wavelength region of the reflected light.

According to the present invention, the aiming light can always be recognized in the observation portion image in the normal light observation mode and the special light observation mode.

### BRIEF DESCRIPTION OF THE DRAWINGS

FIG. 1 is a schematic configuration view of an optical tomographic imaging apparatus;

FIG. 2 is a schematic configuration view of an observation portion image obtaining portion; and

FIG. 3 is a schematic configuration view of an OCT obtaining portion.

### DETAILED DESCRIPTION OF THE PREFERRED EMBODIMENTS

The preferred embodiment of the present invention will be described below in detail with reference to the accompanying drawings. In this embodiment, an optical tomographic imaging apparatus will be described as one example of the invisible light irradiation apparatus.

### [Description of Optical Tomographic Imaging Apparatus]

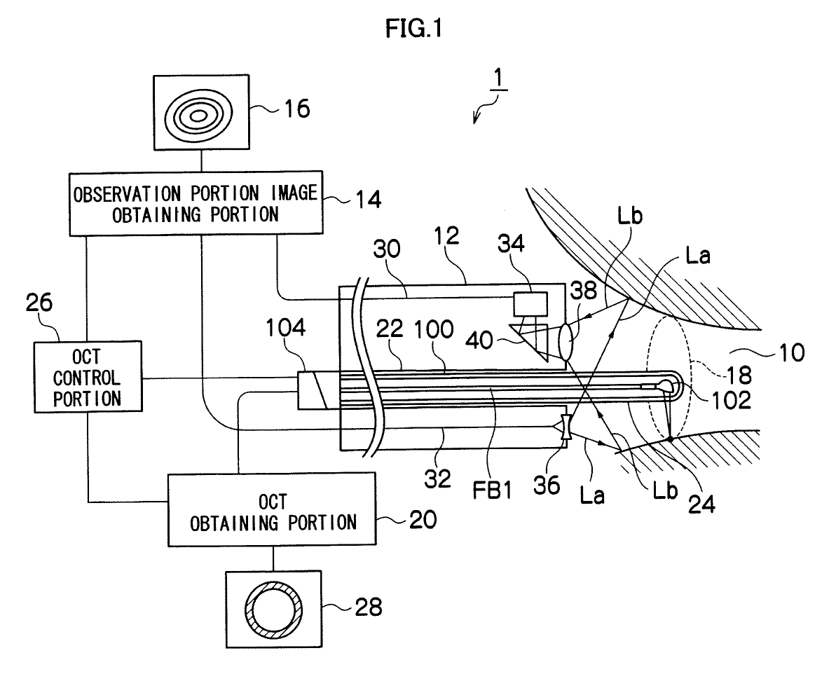

A specific embodiment of the present invention will be described below with reference to the drawings. First, an optical tomographic imaging apparatus which is an embodiment of the present invention will be described with reference to FIG. 1.

The optical tomographic imaging apparatus 1 of the present invention comprises the insertion portion 12 of an endoscope which is inserted into the body cavity 10 of a test subject, an observation portion image obtaining portion 14 which obtains a color image of an observation portion in the body cavity 10 of the living body, and a monitor 16 which displays the color image. Also, the optical tomographic imaging apparatus I of the present invention comprises an OCT obtaining portion 20 which obtains an optical tomographic image of a measurement region 18 in the body cavity 10, an optical probe 24 which is inserted into a forceps port 22 provided in the insertion portion 12 of the endoscope, an OCT control portion 26 which controls an optical tomographic image obtaining operation, and a monitor 28 which displays the optical tomographic image.

The insertion portion 12 comprises the forceps port 22 which passes through the insertion portion 12, a CCD cable 30 which extends inside to a tip, and a light guide 32. A CCD image sensor 34 is connected to the tip of the CCD cable 30. A pickup lens 38 is provided in the tip portion of the insertion portion 12, and a prism 40 is provided on the inside of this pickup lens 38. An illumination lens 36 is provided in the tip portion of the light guide 32, that is, the tip portion of the insertion portion 12. Also, the CCD cable 30 and the light guide 32 are connected to the observation portion image obtaining portion 14.

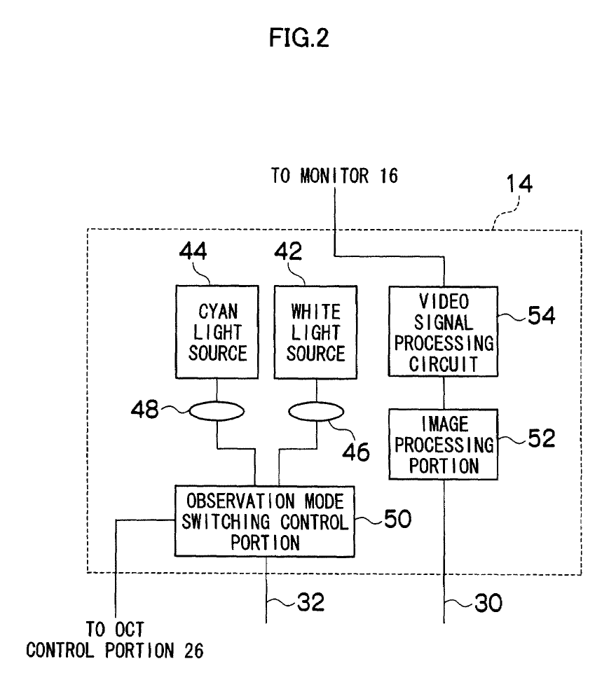

As shown in FIG. 2, the observation portion image obtaining portion 14 comprises a white light source 42 which emits white light which is for observation portion image pickup and is used in a normal light observation mode, and a cyan light source 44 which emits cyan light which is also for observation portion image pickup and is used in a special light observation mode.

Also, a lens 46 is located in an emission portion for the light of the white light source 42, and a lens 48 is located in an emission portion for the light of the cyan light source 44. An observation mode switching control portion 50 (illumination light switching control portion) is further provided in the direction of emission of light from the lens 46 and the lens 48. The observation mode switching control portion 50 is connected to the above OCT control portion 26 and the light guide 32.

Also, the observation portion image obtaining portion 14 comprises, in addition, an image processing portion 52 which is connected to the CCD cable 30 and generates an image signal, based on an image signal picked up by the CCD image sensor 34, and a video signal processing circuit 54 which converts the image signal, output from the image processing portion 52, to a video signal, and outputs the video signal to the monitor 16.

In the optical tomographic imaging apparatus I having such a configuration, an observer inserts the insertion portion 12 into the body cavity 10 of the test subject to display an observation portion image on the monitor 16.

By the control of the observation mode switching control portion 50, in a normal light observation mode, white light emitted from the white light source 42 of the observation portion image obtaining portion 14 enters the light guide 32 by the lens 46, is guided to the tip of the insertion portion 12, and then irradiates the body cavity 10 from the illumination lens 36. Then, the reflected light Lb of white light La is collected by the pickup lens 38, reflected by the prism 40, and imaged on the CCD image sensor 34. An image signal photoelectrically converted by the CCD image sensor 34 is output to the image processing portion 52 via the CCD cable 30.

Also, by the control of the observation mode switching control portion 50, in a special light observation mode, cyan light emitted from the cyan light source 44 of the observation portion image obtaining portion 14 enters the light guide 32 by the lens 48, is guided to the tip of the insertion portion 12, and then irradiates the body cavity 10 from the illumination lens 36. Then, the reflected light Lb of cyan light La is collected by the pickup lens 38, reflected by the prism 40, and imaged on the CCD image sensor 34. An image signal photoelectrically converted by the CCD image sensor 34 is output to the image processing portion 52 via the CCD cable 30.

The cyan light source 44 may be separately provided in the tip portion of the insertion portion 12, rather than in the observation portion image obtaining portion 14.

Also, the observation portion image obtaining portion 14 may comprise only the white light source 42, without the cyan light source 44, and further comprise a cut filter which cuts light in a wavelength region other than that of cyan light. Then, by the control of the observation mode switching control portion 50, in the normal light observation mode, white light from the white light source 42 may be allowed to enter the light guide 32 as it is, without using the cut filter, while in the special light observation mode, only cyan light from white light from the white light source 42 is allowed to enter the light guide 32, using the cut filter.

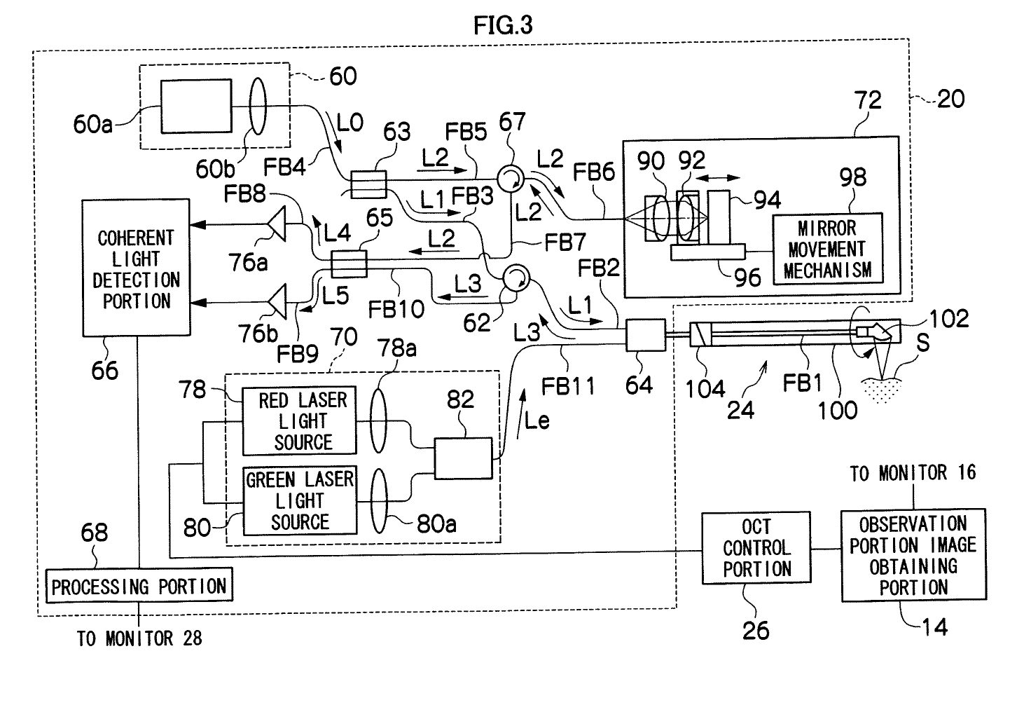

The optical probe 24 is inserted into the forceps port 22 of the insertion portion 12. Then, while an observation portion image displayed on the monitor 16 is observed, the tip of the insertion portion 12 is moved, and when it reaches near a site where an optical tomographic image is obtained, laser light or low coherence light L0 is emitted from the first light source (first light source unit) 60 (see FIG. 3) of the OCT obtaining portion 20.

Next, the OCT obtaining portion 20 will be described.

As shown in FIG. 3, the OCT obtaining portion 20 is to obtain an optical tomographic image of an object to be measured, by optical coherence tomography (OCT) measurement, and has the first light source (first light source unit) 60 which emits light L0 for measurement, an optical fiber coupler (dividing and combining portion) 63 which divides the light L0, emitted from the first light source 60, into measurement light (first light beam) L1 and reference light L2, a WDM coupler 64 which combines aiming light Le described later and the measurement light L1, an optical fiber FB2 which guides light to the WDM coupler 64 and guides return light L3 guided by an optical fiber FB1, an optical fiber coupler 65 which combines the reference light L2, subjected to frequency shift and the change of the optical path length by an optical path length adjustment portion 72 described later and returned, and the return light L3 from an object to be measured S, a coherent light detection portion 66 which detects as coherent signals coherent light L4 and coherent light L5 generated by the optical fiber coupler 65, and a processing portion 68 which processes the coherent signals detected by this coherent light detection portion 66 to obtain an optical tomographic image (hereinafter also simply referred to as a “tomographic image”).

Also, the OCT obtaining portion 20 has a second light source (second light source unit) 70 which emits the aiming light (second light beam) Le for indicating a measurement mark, the optical path length adjustment portion 72 which adjusts the optical path length of the reference light L2, and detection portions 76a and 76b which detect the coherent light L4 and the coherent light L5 generated by the optical fiber coupler 65.

The first light source 60 emits OCT signal light (for example, wavelength-swept laser light having a center wavelength of 1.3 μm or low coherence light) and comprises a light source 60a which emits the laser light or low coherence light L0, and a lens 60b which collects the light L0 emitted from the light source 60a. The light L0 emitted from the first light source 60 is divided into the measurement light L1 and the reference light L2 by the optical fiber coupler 63 via an optical fiber FB4, and the measurement light L1 is input to the WDM coupler 64 via a circulator 62. The optical fiber coupler 63 divides the light, for example, in a ratio of measurement light L1:reference light L2=99:1.

Also, the second light source 70 emits colored light with visibility as the aiming light Le so that a measurement site is easily recognized. In this embodiment, the second light source 70 has a red laser light source 78 which emits red laser light (for example, laser light having a wavelength of 630 nm), and a green laser light source 80 which emits green laser light (for example, laser light having a wavelength of 532 nm).

Also, the second light source 70 has a lens 78a which collects the aiming light Le emitted from the red laser light source 78, and a lens 80a which collects the aiming light Le emitted from the green laser light source. Then, the aiming light Le emitted from the lens 78a and the lens 80a enters a WDM coupler 82, and then is input to the WDM coupler 64 via an optical fiber FB11. In the WDM coupler 64, the measurement light L1 and the aiming light Le are combined and guided to the optical fiber FB1 in the optical probe 24.

Also, the red laser light source 78 and the green laser light source 80 are connected to the OCT control portion 26 which also serves as a guide light control portion, and their operation is controlled by the OCT control portion 26.

The circulator 62 allows the measurement light L1, which enters from the optical fiber coupler 63 via an optical fiber FB3, to enter the optical fiber FB2, and allows the return light L3 from the object to be measured S to enter an optical fiber FB10.

A circulator 67 allows the reference light L2, which enters from the optical fiber coupler 63 via an optical fiber FB5, to enter an optical fiber FB6, and emits the reference light L2, which is subjected to frequency shift and the change of the optical path length by the optical path length adjustment portion 72 described later and returned through the optical fiber FB6, to an optical fiber FB7.

The optical probe 24 is connected to the optical fiber FB2 via the WDM coupler 64, and the measurement light L1 combined with the aiming light Le enters the optical fiber FB1 from the optical fiber FB2 via the WDM coupler 64. This entering measurement light L1 combined with the aiming light Le is transmitted by the optical fiber FB1 and irradiates the object to be measured S. Then, the return light L3 from the object to be measured S is obtained, and the obtained return light L3 is transmitted by the optical fiber FB1 and emitted to the optical fiber FB2 via the WDM coupler 64.

The coherent light detection portion 66 is connected to an optical fiber FB8 and an optical fiber FB9 and detects as coherent signals the coherent light L4 and the coherent light L5 generated by combining the reference light L2 and the return light L3 by the optical fiber coupler 65.

The processing portion 68 obtains a tomographic image from the coherent signals detected by the coherent light detection portion 66.

The optical path length adjustment portion 72 is located on the reference light L2 emission side of the optical fiber FB6.

The optical path length adjustment portion 72 has a first optical lens 90 which turns the reference light L2, emitted from the optical fiber FB6, into parallel light, a second optical lens 92 which collects the light turned into parallel light by the first optical lens 90, a reflecting mirror 94 which reflects the light collected by the second optical lens 92, a base 96 which supports the second optical lens 92 and the reflecting mirror 94, and a mirror movement mechanism 98 which moves the base 96 in a direction parallel to the direction of the optical axis. The optical path length adjustment portion 72 adjusts the optical path length of the reference light L2 by changing the distance between the first optical lens 90 and the second optical lens 92.

The optical probe 24 comprises a sheath 100 which can be inserted into the forceps port 22 of the insertion portion 12 (see FIG. 1) and rotated, the optical fiber FB I which passes through the sheath 100, and a collecting and reflecting lens system 102 which is provided in the sheath 100 tip direction of the optical fiber FB1. The tip portion of the sheath 100 is transparent. Also, a rotary joint 104 which rotates the sheath 100 in the radial direction (circumferential direction) and slides the sheath 100 in the linear direction (axial direction) is attached to the base portion of the sheath 100.

The OCT control portion 26 is connected to each site of the OCT obtaining portion 20 and appropriately controls the operation timing of each site. Also, the OCT control portion 26 controls the operation of the rotary joint 104 to control the rotation of the direction of irradiation of the measurement light L1 and the position of irradiation of the measurement light L1.

### [Description of Switching of Aiming Light]

The switching of the aiming light Le in the optical tomographic imaging apparatus 1 of the present invention having the configuration and basic action as described above will be described.

In the optical tomographic imaging apparatus I of the present invention, the red laser light source 78 and the green laser light source 80 are located in the second light source 70 in the OCT obtaining portion 20, as shown in the above FIG. 3, and laser light sources for a plurality of aiming lights Le in different wavelength regions are located.

Also, the white light source 42 which is a light source for illumination light in the normal light observation mode, and the cyan light source 44 which is a light source for illumination light in the special light observation mode are located in the observation portion image obtaining portion 14 connected to the light guide 32, as shown in the above FIG. 2, and light sources for a plurality of illumination lights in different wavelength regions are located.

Then, by switching the wavelength region of the aiming light Le between when an object to be measured is irradiated with illumination light in the normal light observation mode and when the object to be measured is irradiated with illumination light in the special light observation mode, the visibility of the position of measurement with the aiming light Le is improved in an observation portion image in the monitor 16.

Specifically, first, in the normal light observation mode in which white light is irradiated from the white light source 42 as illumination light, the emission of red laser light from the red laser light source 78 is turned to OFF, while the emission of green laser light from the green laser light source 80 is turned to ON, in the second light source 70 in the OCT obtaining portion 20.

Thus, in the normal light observation mode, for example, when the object to be measured is a red gastric mucosa, the periphery of the position of measurement is displayed in red on the monitor for an observed image 16 by the illumination light, but the green aiming light Le is irradiated, so that the visibility of the position of irradiation of the aiming light Le is improved in the observation portion image in the monitor 16. Therefore, an observer can easily recognize the position of measurement on the monitor for an observed image 16.

Here, when the normal light observation mode is switched to the special light observation mode in which cyan light is irradiated from the cyan light source 44 as illumination light, in response to this, a trigger signal is sent from the observation mode switching control portion 50 of the observation portion image obtaining portion 14 to the OCT control portion 26. Then, the OCT control portion 26 controls so that the emission of red laser light from the red laser light source 78 is turned to ON, while the emission of green laser light from the green laser light source 80 is turned to OFF, in the second light source 70.

Thus, in the special light observation mode, the periphery of the position of measurement is displayed in cyan in the observation portion image in the monitor 16 by cyan illumination light, but the red aiming light Le is irradiated, so that the visibility of the position of irradiation of the aiming light Le is improved in the observation portion image in the monitor 16. Particularly, red is the complementary color of cyan, so that the visibility is especially improved. Therefore, also in the special light observation mode, the observer can easily recognize the position of measurement on the monitor for an observed image 16, as in the normal light observation mode.

When the special light observation mode is switched to the normal light observation mode again, in response to this, a trigger signal is sent from the observation mode switching control portion 50 of the observation portion image obtaining portion 14 to the OCT control portion 26. Then, the OCT control portion 26 controls so that the emission of red laser light from the red laser light source 78 is turned to OFF, while the emission of green laser light from the green laser light source 80 is turned to ON, in the second light source 70.

Then, similarly, for each switching between the normal light observation mode and the special light observation mode, a trigger signal is sent from the observation mode switching control portion 50 of the observation portion image obtaining portion 14 to the OCT control portion 26, and the OCT control portion 26 performs control to switch the ON and OFF of the emission of red laser light from the red laser light source 78 and the ON and OFF of the emission of green laser light from the green laser light source 80 in the second light source 70.

In this manner, the wavelength region of the aiming light Le is switched corresponding to the wavelength region of the illumination light in the normal light observation mode and the special light observation mode, so that the aiming light can always be visually recognized in the observation portion image in the normal light observation mode and the special light observation mode.

For switching between the emission of red laser light from the red laser light source 78 and the emission of green laser light from the green laser light source 80, a shutter may be used.

Also, instead of the WDM coupler 82 to which red laser light and green laser light are input, a configuration in which red laser light and green laser light can be spatially combined using a dichroic mirror may be used.

Also, switching between the emission of red laser light and the emission of green laser light may be performed by using a switching device instead of the WDM coupler 82 to which red laser light and green laser light are input. At this time, the OCT control portion 26 controls the emission of red laser light from the red laser light source 78 and the emission of green laser light from the green laser light source 80 to be always ON, and the switching device performs switching between the entry of red laser light and the entry of green laser light into the WDM coupler 64 which combines the measurement light L1 and the aiming light Le.

Also, in this embodiment, the red laser light source 78 and the green laser light source 80 are used in the second light source 70, but the laser light source is not limited to these. For example, a blue laser light source may be used instead of the green laser light source 80. In addition, a source of laser light of a color corresponding to the complementary color of the color of illumination light in the normal light observation mode and the special light observation mode may be used.

As described above, in this embodiment, the red laser light source 78 and the green laser light source 80 which emit the aiming light Le are provided, and control is performed to switch the aiming light Le to red laser light or green laser light according to the observation mode, so that the visibility of the aiming light Le is improved according to the observation mode, and the aiming light can always be recognized in the observation portion image on the monitor 16, regardless of the observation mode.

### [Modifications]

As a modification, an optical tomographic imaging apparatus is also considered in which a cut filter which cuts light in a specific wavelength region is located on the side of a CCD mechanism composed of the pickup lens 38, the prism 40, the CCD image sensor 34, the CCD cable 30, and the like, and an observation portion image in which light in the specific wavelength region is cut is displayed on the monitor 16 using the cut filter by the received light switching control portion (not shown) of the observation portion image obtaining portion 14 in a special light observation mode.

In the case of this optical tomographic imaging apparatus, the emission of red laser light and the emission of green laser light are always performed from the second light source 70, and the aiming light Le, both the red laser light and the green laser light, is combined with the measurement light L1. Thus, even if either one color light of the red light and the green light is cut by the cut filter, the position of irradiation of the aiming light Le can always be visually recognized on the monitor 16 with the other color light.

To reduce electric power and prevent excessive light irradiation of a living body, it is desired that for each switching between the normal light observation mode and the special light observation mode, a trigger signal is sent from the received light switching control portion to the OCT control portion 26, and the OCT control portion 26 controls the second light source 70 to switch the wavelength region of laser light according to the mode so that laser light in an unnecessary wavelength region is not emitted.

Also, in an optical tomographic imaging apparatus in which the observation of a living body mucosa is performed by an infrared electronic scope in which infrared observation is possible, it is considered that other than visible laser light sources, such as the red laser light source 78 and the green laser light source 80, an infrared laser light source which emits infrared laser light is also provided in the second light source 70. The infrared electronic scope uses a CCD which is sensitive to a wavelength of 790 to 970 nm. OCT measurement light having a wavelength of 1300 nm is not sensitive to this infrared electronic scope, so that it also cannot be visually recognized. Therefore, a source of infrared laser light having a wavelength of 790 to 970 nm to which the infrared electronic scope is sensitive is desired in a special light observation mode.

When the normal light observation mode is switched to the special light observation mode in which infrared light is used as illumination light to display an infrared image on the monitor for an observation portion image 16, the OCT control portion 26 controls so that the emission of infrared laser light from the infrared laser light source is turned to ON in the second light source 70.

Thus, also in the special light observation mode in which infrared light is used as illumination light, the position of irradiation of the aiming light Le can be visually recognized in an observation portion image, which is an infrared image, displayed on the monitor 16.

Also, in an observation method using self-fluorescence from a living body tissue, in a special light observation mode in which an object to be measured is irradiated with light in a wavelength region close to that of ultraviolet light, as illumination light, to emit green light, and an observation portion image in which ultraviolet light is cut using an ultraviolet light cut filter is obtained to perform observation, when the OCT control portion 26 controls so that the emission of red laser light from the red laser light source 78 is turned to ON, while the emission of green laser light from the green laser light source 80 is turned to OFF, in the second light source 70, the position of irradiation of the aiming light Le can be visually recognized in an observation portion image displayed on the monitor for an observed image 16.

In addition, in an observation method in which a fluorescent substance is sprayed over or injected into an affected part to observe the distribution of fluorescence selectively emitting light in the affected part, in a special light observation mode in which illumination light having a wavelength suited to each fluorescent substance, and a transmission filter are used, the position of irradiation of the aiming light Le can be visually recognized by selecting the wavelength of laser light suited to each mode.

The invisible light irradiation apparatus and the method for controlling the invisible light irradiation apparatus according to the present invention have been described in detail, but the present invention is not limited to the above examples. Of course, various improvements and modifications may be made without departing from the gist of the present invention.

For example, the invisible light irradiation apparatus is not limited to the optical tomographic imaging apparatus, and an invisible light irradiation apparatus in which the optical axis of invisible light is scanned using the probe as described above can also be applied to a laser light irradiation treatment apparatus and the like.

What is claimed is:

1. An invisible light irradiation apparatus comprising:
an invisible light source which emits invisible light for irradiating an object to be irradiated; and
a guide light source which emits guide light for being combined with the invisible light to irradiate the object to be irradiated to clearly indicate a position of irradiation of the invisible light,
wherein a plurality of the guide light sources which emit the guide light in different wavelength regions are provided.

2. The invisible light irradiation apparatus according to claim 1, further comprising:
an illumination device which emits illumination light used for observing the object to be irradiated and emits a plurality of the illumination lights in different wavelength regions;
an illumination light switching control portion which switches the wavelength region of the illumination light to switch an observation mode for observation of the object to be irradiated; and
a guide light control portion which switches the wavelength region of the guide light,
wherein the guide light control portion switches the wavelength region of the guide light according to the observation mode switched by the illumination light switching control portion.

3. The invisible light irradiation apparatus according to claim 2, wherein the guide light control portion switches the wavelength region of the guide light to be different from the wavelength region of the illumination light.

4. The invisible light irradiation apparatus according to claim 2, wherein the guide light control portion switches the wavelength region of the guide light so that a color of the guide light is a complementary color for a color of the illumination light.

5. The invisible light irradiation apparatus according to claim 3, wherein the guide light control portion switches the wavelength region of the guide light so that a color of the guide light is a complementary color for a color of the illumination light.

6. The invisible light irradiation apparatus according to claim 2, wherein the guide light control portion switches the wavelength region of the guide light so that the guide light is infrared light when the illumination light is infrared light.

7. The invisible light irradiation apparatus according to claim 3, wherein the guide light control portion switches the wavelength region of the guide light so that the guide light is infrared light when the illumination light is infrared light.

8. The invisible light irradiation apparatus according to claim 1, further comprising:
an illumination device which emits illumination light used for observing the object to be irradiated;
a received light switching control portion which switches a wavelength region for receiving reflected light from the object to be irradiated to switch an observation mode for the object to be irradiated; and
a guide light control portion which switches the wavelength region of the guide light,
wherein the guide light control portion switches the wavelength region of the guide light according to the observation mode switched by the received light switching control portion.

9. The invisible light irradiation apparatus according to claim 1, further comprising:
an elongated insertion portion which can be inserted into the object to be irradiated;
a light guide device which is inserted into the insertion portion, guides the invisible light to irradiate the object to be irradiated from a tip portion of the insertion portion, and guides reflected light reflected by the object to be irradiated;
a coherent light extraction device which allows the reflected light guided by the light guide device and the invisible light to interfere with each other to extract a coherent signal corresponding to interfering coherent light; and
a signal processing device which performs signal processing corresponding to the coherent signal and generates a tomographic image of the object to be irradiated in a depth direction.

10. A method for controlling an invisible light irradiation apparatus, comprising:
combining invisible light for irradiation of an object to be irradiated with guide light;
irradiating the object to be irradiated with the combined lights to clearly indicate a position of irradiation of the invisible light by the guide light; and
emitting illumination light for observing the object to be irradiated,
wherein a wavelength region of the guide light is switched according to a wavelength region of the illumination light.

11. A method for controlling an invisible light irradiation apparatus, comprising:
combining invisible light for irradiation of an object to be irradiated with guide light;
irradiating the object to be irradiated with the combined lights to clearly indicate a position of irradiation of the invisible light by the guide light; and
receiving reflected light from the object to be irradiated,
wherein a wavelength region of the guide light is switched according to a wavelength region of the reflected light.
